(19) United States
(12) Patent Application Publication
(10) Pub. No.: US 2014/0316276 A1
(43) Pub. Date: Oct. 23, 2014

(54) BACKING ELEMENT AND ULTRASOUND PROBE INCLUDING SAME

(71) Applicants: Byungkuk Bae, Daegu (KR); Susung Lee, Kyunggi-do (KR)

(72) Inventors: Byungkuk Bae, Daegu (KR); Susung Lee, Kyunggi-do (KR)

(73) Assignee: ALPINION MEDICAL SYSTEMS CO., LTD., Hwaseong-si, Gyeonggi-do (KR)

(21) Appl. No.: 14/369,546

(22) PCT Filed: Dec. 30, 2011
(86) PCT No.: PCT/KR2011/010389

§ 371 Date: Jun. 27, 2014

(30) Foreign Application Priority Data
Dec. 30, 2011 (KR) 10-2011-0147026

(51) Int. Cl. A61B 8/00

(52) U.S. Cl. CPC A61B 8/4444; A61B 8/4272
USPC 600/472; 600/459

(57) ABSTRACT

The present disclosure in some embodiment provides a backing member for an ultrasonic probe that is disposed on a rear surface of a piezoelectric element constituting the ultrasonic probe to attenuate ultrasonic waves radiated from the rear surface of the piezoelectric element, the backing member including a mixture of a base member and a filler, wherein the base member is a polyurethane resin and the filler is manganese powder, whereby the backing member has appropriate sound absorption function and excellent dicing processability and provides an ultrasonic probe including the backing member.

### TECHNICAL FIELD

The present disclosure in one or more embodiments relates to a backing member used in an ultrasonic probe. More particularly, the present disclosure relates to a backing member having excellent dicing processability and appropriate sound absorption performance that attenuates unnecessary ultrasonic waves radiated from a rear surface of a piezoelectric element constituting an ultrasonic probe and an ultrasonic probe including the backing member.

### BACKGROUND

Ultrasonic diagnostic apparatus or ultrasonic image inspection apparatus for medical purposes performs imaging of an internal structure of a target object by transmitting an ultrasonic signal to an object under examination and receiving an ultrasonic echo signal therefrom. Such ultrasonic diagnostic apparatus or ultrasonic image inspection apparatus mainly employs an array ultrasonic probe capable of transmitting/receiving ultrasonic signals.

FIG. 1 is a partial perspective view of an ultrasonic probe. As illustrated in FIG. 1, the ultrasonic probe 1 includes a support body 2 and a sheet-shaped backing member 3 that is fixedly adhered to the support body 2 through an adhesive.

A piezoelectric element 4 is adhesively fixed to the backing member 3. The piezoelectric element 4 includes a piezoelectric vibrator, a ground electrode disposed at a front surface of the piezoelectric vibrator, and a signal electrode disposed at a rear surface of the piezoelectric vibrator.

An acoustic matching layer 5 is adhesively fixed to the ground electrode of the piezoelectric element 4 and thus can reduce a difference in acoustic impedance between the piezoelectric element 4 and an object under examination (not shown).

An acoustic lens 6 is fixedly adhered to the acoustic matching layer 5 through an adhesive.

Hereinafter, an assembly including the backing member 3, piezoelectric element 4, acoustic matching layer 5 and acoustic lens 6 is referred to as an “array.” The array is accommodated in a case 7 and fixedly adhered to the support body 2. In addition, the case 7 may accommodate a signal processing circuit (not shown) including a control circuit for controlling driving timing of the piezoelectric element 4 and an amplification circuit for amplifying signals received by the piezoelectric element 4.

A cable 8 that is connected to the ground and signal electrodes of the piezoelectric element 4 extends external to the case 7 at the opposite side of the acoustic lens 6.

In the ultrasonic probe 1 having such configuration, the piezoelectric vibrator vibrates at each of a plurality of channels by application of voltage between the ground and signal electrodes of the piezoelectric element 4 and, consequently, ultrasonic waves are emitted via the acoustic matching layer 5 and the acoustic lens 6. Upon receiving reflected ultrasonic echo signals, the piezoelectric vibrator of the piezoelectric element 4 vibrates according to ultrasonic signals received via the acoustic lens 6 and the acoustic matching layer 5 and the piezoelectric element 4 converts this vibration into an electrical signal to be used in acquiring an image.

When the piezoelectric element 4 is driven, ultrasonic signals are emitted from both the front and rear surfaces thereof. For this reason, the backing member 3 is disposed on the rear surface of the piezoelectric element 4 to absorb and attenuate the ultrasonic signals emitted from the rear surface thereof and to prevent a simultaneous transmission of normal ultrasonic signals and the ultrasonic (echo) signals emitted from the rear surface of the piezoelectric element 4 into an object under examination.

Ultrasonic probes employed in ultrasonic diagnostic apparatuses and the like require relatively high sensitivity to achieve a high image quality. Acquiring the high sensitivity needs enhancement of transmitting capability and receiving sensitivity of ultrasonic waves. A method for decreasing an acoustic impedance of a backing member can be a solution.

Decrease in an acoustic impedance of the backing member not only attenuates ultrasonic waves emitted towards the backing member and efficiently transmit ultrasonic waves from an ultrasonic wave transmission/reception wave surface of a front surface of an ultrasonic probe but also enhances the sensitivity of received waves due to decrease in acoustic impedance of a wave-receiving system.

As an example, in an ultrasonic probe for an ultrasonic diagnostic apparatus, the probe sensitivity is enhanced with an assist from a backing member having an acoustic impedance of about 2 to 5 MRayls. However, a backing member having an acoustic impedance of about 2 to 5 MRayls, which is necessary to achieve a high sensitivity, needs to have a sufficient hardness and a sound absorption capability. However, it has been difficult to obtain materials that satisfy these needs.

With a backing member of poor acoustic property, ultrasonic waves cannot be sufficiently absorbed and attenuated and, accordingly, ultrasonic image quality may be deteriorated. In particular, the acoustic impedance of a backing member is a design choice and a good backing member would have a uniform acoustic impedance throughout. In other words, it is necessary to adjust the acoustic impedance uniform by setting attenuation of the backing member to desired values.

Conventionally, a mixture of rubber and ferrite powder is used as a backing member. However, such a backing member has exhibited difficulty in enhancing ultrasonic wave attenuation performance. In addition, when used for manufacturing a high-frequency ultrasonic probe, the backing member is too soft for dicing with a dice having a fine pitch. Further, when dicing is applied, the backing member is easily subject to deformation by heat. Moreover, when a rubber material is used, it is difficult to adjust acoustic impedance and attenuation. In particular, rubber material having the acoustic impedance as low as about 2 to 5 MRayls is hardly available.

Meanwhile, it is known backing members are fabricated by mixing an epoxy resin and powder of tungsten or the like as a filler. Such backing members may have high rigidity and high attenuation performance.

In this case, however, a filler such as tungsten or the like has very high mass per unit volume (density) and thus, when powder thereof is mixed with an epoxy resin or the like, the powder precipitates downward and the number of particles per unit volume in a vertical direction (thickness direction) becomes non-uniform. In other words, it is difficult to obtain a uniform acoustic property (in particular, acoustic impedance) for a backing member in the vertical direction. In addition, such difficulty made it hard to obtain a high level of attenuation.

In addition, the backing member formed of a mixture of an epoxy resin and powder of tungsten or the like, having the excessive hardness of tungsten, makes dicing difficult. In particular, where the backing member includes 10 to 100 wt % of tungsten powder having particle sizes of several to tens of micrometers and the epoxy resin which is soft, cutting from an acoustic matching layer through a piezoelectric member and the backing member with a dice in order to form channels generates stress for causing fracture or separation between the epoxy resin and the filler. Some stresses when cutting the backing member tend to bring about even the bending of backing member, separation between the backing member and the piezoelectric element, or the like.

### DISCLOSURE

### Technical Problem

Therefore, the present disclosure has been made to provide a backing member having a novel composition of a mixture of a polyurethane resin and manganese powder to achieve excellent dicing processability and appropriate sound absorption performance that attenuates unnecessary ultrasonic waves radiated rearward of a piezoelectric element constituting an ultrasonic probe and an ultrasonic probe including the same.

### SUMMARY

In accordance with some embodiments of the present disclosure, a backing member for an ultrasonic probe being arranged on a rear surface of a piezoelectric element employed in the ultrasonic probe to attenuate ultrasonic waves radiated from the rear surface of the piezoelectric element includes a mixture of a base member and a filler, wherein the base member is a polyurethane resin and the filler is manganese powder.

In accordance with some embodiments of the present disclosure, an ultrasonic probe includes the backing member described above.

### Advantageous Effects

According to the present disclosure as described above, there are provided a backing member having appropriate sound absorption performance and excellent dicing processability and an ultrasonic probe including the same.

In addition, according to the present disclosure as described above, a heat transfer rate is additionally increased and, accordingly, heat dissipation performance is enhanced and thus an ultrasonic probe may be driven at high voltage.

Ultimately, according to the present disclosure as described above, a defect rate of the backing member is reduced and production yield is significantly increased, and product salability is enhanced.

### DESCRIPTION OF DRAWINGS

FIG. 1 is a partial perspective view of a ultrasonic probe.

FIG. 2 is a schematic view of a system for measurement of attenuation performance of a backing member according to the present disclosure and for comparison between the backing member and a conventional backing member.

FIG. 3 is a graph showing an attenuation constant obtained from experimental results of attenuation performance of backing member specimens having different thicknesses.

FIG. 4 is a diagram illustrating a state of the backing member according to the present disclosure after dicing.

FIG. 5 is a diagram illustrating a state of a conventional backing member after dicing.

### DETAILED DESCRIPTION

Hereinafter, at least one embodiment of the present disclosure will be described in detail with reference to the accompanying drawings. In the following description of the at least one embodiment, a detailed description of known functions and configurations incorporated herein will be omitted for the purpose of clarity and brevity.

A backing member for ultrasonic probes according to the present disclosure is configured to attenuate ultrasonic waves emitted from a rear surface of a piezoelectric element employed in an ultrasonic probe. The backing member includes a mixture of a base member or material and a filler. The base member is a polyurethane resin and the filler is manganese powder.

The term “rear surface of a piezoelectric element” as used herein represents a space residing opposite to a (front) side at which the piezoelectric element transmits and receives ultrasonic waves.

The backing member may have a high heat conductivity to avoid increase in temperature of a surface (at an acoustic lens) of an ultrasonic probe and is rather of small size and lightweight. To fabricate a small and lightweight backing member, it is suitably formed of a material having high ultrasonic wave attenuation performance and low density. Thus, the backing member is suitably formed of a material having an acoustic impedance of about 2 to 5 MRayls. In addition, it is important that the backing member has insulation property.

The backing member is an ultrasonic wave absorber for supporting a piezoelectric element that serves as an ultrasonic vibrator of an ultrasonic probe and for absorbing unnecessary ultrasonic waves. The backing member is manufactured from a mixture of a base member and a filler.

The base member may be of a polyurethane resin. A single type of polyurethane resin may be used while two or more kinds of polyurethane resin may be used in combination.

A polyurethane resin composition may be obtained by mixing a liquid-phase prepolymer and a liquid-phase curing agent for polyurethane resins. In the liquid-phase polyurethane resin composition, the amount of the curing agent may be about 60 parts by weight based on 100 parts by weight of the prepolymer.

The relevant filler to the present disclosure may, for example, be manganese powder. The manganese powder may have an average particle diameter of 100 mesh or larger diameter, in particular about 80 mesh.

As such, the backing member according to the present disclosure has a structure with the base of a polyurethane resin which is filled with a powder material such as manganese powder.

Meanwhile, an experiment is conducted for measuring attenuation performance of the backing member described above.

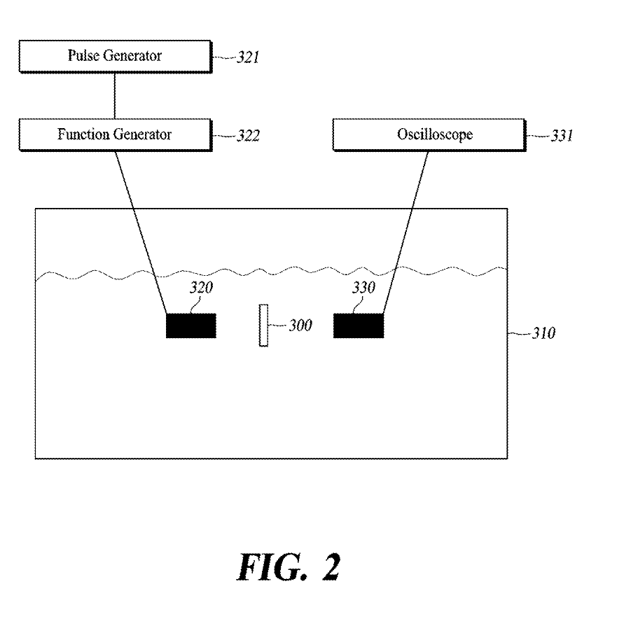

FIG. 2 is a schematic view of a system for measurement of attenuation performance of a backing member according to the present disclosure and for comparison between the backing member and a conventional backing member.

The system illustrated in FIG. 2 is configured to measure attenuation performance of the backing member by using a Through Transmission Method, in which two identical ultrasonic probes 320 and 330 face each other with a backing member specimen 300 disposed therebetween in a water tank 310 filled with water so that the ultrasonic probe 320 performs a transmission function and the ultrasonic probe 330 performs a reception.

To simulate a living body, the water tank 310 is filled with water of about 38° C.

The ultrasonic probes 320 and 330 used are manufactured by Panametrics™ and have a frequency of 3.5 MHz and a diameter of 1 inch (i.e., about 25 mm).

In addition, a pulse generator 321 and a function generator 322 are connected to the transmitting ultrasonic probe 320 to which 5V is applied. Meanwhile, the receiving ultrasonic probe 330 is connected to an oscilloscope 331.

As the backing member specimen 300, backing member specimens according to the present disclosure and conventional backing member specimens are prepared.

As the backing member specimens according to the present disclosure, Specimen 1 and Specimen 2 are prepared.

Specimen 1 is fabricated by mixing 47 wt % of a liquid-phase polyurethane resin composition and 53 wt % of manganese powder, degassing the mixture, molding the mixture into a backing member block having a predetermined thickness, and curing the molded block. For example, backing member blocks having respective thicknesses of 2.01 mm, 2.49 mm and 2.95 mm are fabricated and acoustic properties of each backing member block are evaluated.

Specimen 2 is fabricated by mixing 20 wt % of a liquid-phase polyurethane resin composition and 80 wt % of manganese powder, degassing the mixture, molding the mixture into a backing member block having a predetermined thickness, and curing the molded block. Similarly, backing member blocks having respective thicknesses of 2.01 mm, 2.49 mm and 2.95 mm are fabricated and acoustic properties of each backing member block are evaluated.

As the conventional backing member specimens, Specimen 3 and Specimen 4 are prepared.

Specimen 3 is fabricated in the form of a backing member block having a predetermined thickness by mixing 65 wt % of an epoxy resin composition and 35 wt % of tungsten powder. For example, backing member blocks having respective thicknesses of 2.01 mm, 2.49 mm and 2.95 mm are fabricated and acoustic properties of each backing member block are evaluated.

Specimen 4 is fabricated in the form of a backing member block having a predetermined thickness by mixing 31 wt % of an epoxy resin composition and 69 wt % of tungsten powder. As with Specimen 3, backing member blocks having respective thicknesses of 2.01 mm, 2.49 mm and 2.95 mm are fabricated and acoustic properties of each backing member block are evaluated.

Measurement results of acoustic properties such as attenuation performance obtained by using these backing member specimens are shown in Table 1 below.

TABLE 1

| | Backing member according to | | | |
|---|---|---|---|---|
| | present disclosure | | Conventional backing member | |
| | Specimen | Specimen | Specimen | Specimen |
| | 1 | 2 | 3 | 4 |
| Amount | 53 | 80 | 35 | 69 |
| of filler | (manga- | (manga- | (tung- | (tung- |
| (wt %) | nese) | nese) | sten) | sten) |
| Size of | 80 mesh | 80 mesh | 15 μm | 15 μm |
| filler | | | | |
| Density | 2.036 | 3.596 | 1.712 | 3.225 |
| (g/cm³) | | | | |
| Sound | 1.511 | 1.434 | 1.815 | 1.702 |
| velocity | | | | |
| @ 3.5 MHz | | | | |
| (mm/μs) | | | | |
| Attenuation | 10.59 | 9.27 | 11.01 | 30.09 |
| constant @ | | | | |
| 3.5 MHz | | | | |
| (dB/mm) | | | | |
| Impedance | 3.08 | 5.16 | 3.12 | 5.49 |
| (Mrayl) | | | | |

Ultrasonic sound velocity is measured by using a sound velocity measurement device at about 38° C. and acoustic impedance is obtained by multiplying the measured sound velocity by density.

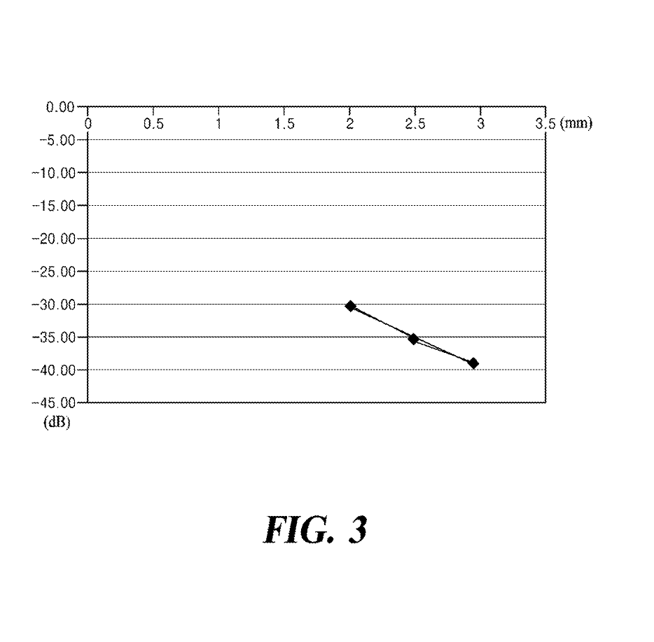

In addition, attenuation performance measurements resulting from using Specimen 2 is illustrated in FIG. 3. For example, backing member blocks having thicknesses of 2.01 mm, 2.49 mm and 2.95 mm, respectively exhibit ultrasonic signal amplitudes of 0.030547 mV, 0.017187 mV and 0.011211 mV at 3.5 MHz. With respect to these results, each amplitude value is calculated by using 20 log₁₀ and then an attenuation constant may be obtained from the log values for the respective thicknesses. Specimen 2 has an attenuation constant of 9.27 dB/mm. In general, an attenuation constant of 9 dB/mm or more is known to be classified as excellent.

Referring to Table 1, the backing members fabricated by using manganese as a filler instead of using tungsten (conventionally used) do not show any major difference in acoustic properties. In particular, even when using a backing member with manganese applied as a filler, the acoustic impedance may be obtained as low as about 2 to 5 MRayls which is needed to realize high sensitivity, and the proportion of manganese may be adjusted so that the backing member according to the present disclosure has appropriate acoustic impedance in accordance with design requirements. For example, the proportion of manganese powder may suitably be in the range of 40 wt % to 90 wt % to obtain an acoustic impedance of about 3 to 5 MRayls. If the proportion of the manganese powder exceeds the above-described range, impedance matching is deteriorated and sound absorption performance of the backing member is degraded.

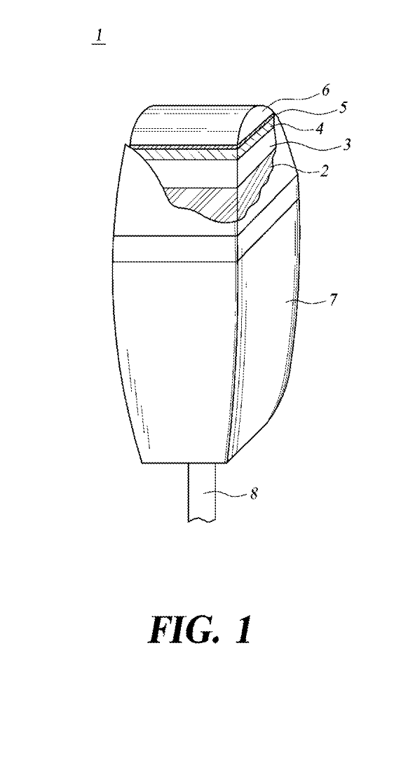

The backing member according to the present disclosure may be applied to ultrasonic probes. Referring back to FIG. 1, the backing member having a sheet shape is attached to the support body 2 through an adhesive or through screw fastening. In addition, the piezoelectric element 4 and the acoustic matching layer 5 may be divided by, for example, dicing processing into arrays and a plurality of channels are formed therein. At the same time, the backing member may have grooves corresponding to the channels.

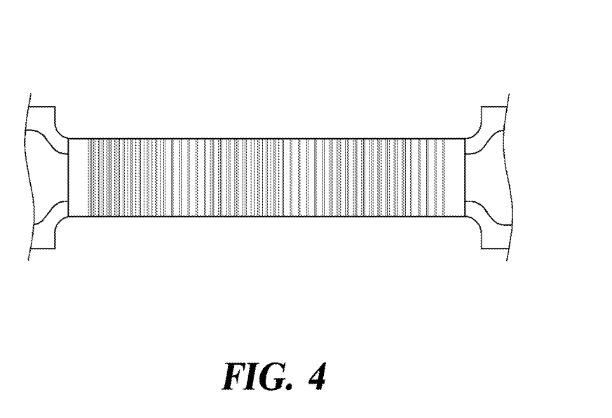

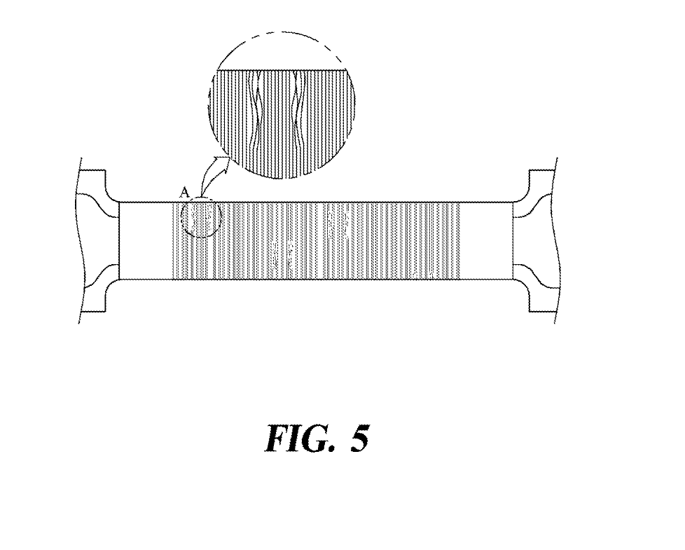

As illustrated in FIG. 4, the backing member according to the present disclosure is subject to dicing into a pitch of approximately 0.2 mm. After the dicing, visual checking with naked eye on the backing member according to the present disclosure exhibits no bending, separation from the piezoelectric member, or the like but overall good quality, when compared to a conventional backing member illustrated in FIG. 5, i.e., a backing member fabricated by mixing an epoxy resin and tungsten powder as a filler. Referring to FIG. 5, it can be confirmed that, as illustrated in an enlarged view of portion A, when dicing is performed, the conventional backing member exhibits fracture or separation between the epoxy resin and the filler by a generated stress.

Therefore, the backing member according to the present disclosure fabricated by mixing a polyurethane resin and manganese powder as a filler ensures higher dicing processability than the conventional backing member fabricated by mixing an epoxy resin and tungsten powder as a filler. Thanks to such enhanced processability, the backing member according to the present disclosure has a reduced defect, a significantly increase in yield, and improved productivity and is manufactured at reduced manufacturing cost. Accordingly, product salability is enhanced.

Meanwhile, in the backing member according to the present disclosure, manganese powder is used in an amount three times or greater than that of conventionally used tungsten powder since the density of the manganese powder is lower than that of the tungsten powder. By such composition, metal components in the backing member are increased and thus heat conductivity thereof is enhanced. Such enhanced heat conductivity in turn increases capabilities of effectively venting heat generated from a piezoelectric element having a plurality of channels and heat accompanying the attenuation of ultrasonic waves of the backing member. Accordingly, an ultrasonic probe may be driven at advantageously high voltage.

The backing member according to the present disclosure is very suitable for use in ultrasonic probes and, when performing imaging of an object under examination, the ultrasonic probe can acquire a clearer image with higher sensitivity.

An ultrasonic probe according to the present disclosure may also be used in ultrasonic diagnostic apparatuses for medical applications, ultrasonic converters for military applications, and ultrasonic apparatuses for industrial applications.

Although exemplary embodiments of the present disclosure have been described for illustrative purposes, those skilled in the art will appreciate that various modifications, additions and substitutions are possible, without departing from the essential characteristics of the disclosure. Therefore, exemplary embodiments of the present disclosure have been described for the sake of brevity and clarity. Accordingly, one of ordinary skill in the art will understand the scope of the disclosure is not limited by the explicitly described above embodiments but by the claims and equivalents thereof.

### CROSS-REFERENCE TO RELATED APPLICATION

If applicable, this application claims priority under 35 U.S.C §119(a) of Patent Application No. 10-2011-0147026, filed on Dec. 30, 2011 in Korea, the entire content of which is incorporated herein by reference. In addition, this non-provisional application claims priority in countries, other than the U.S., with the same reason based on the Korean patent application, the entire content of which is hereby incorporated by reference.

1. A backing member for an ultrasonic probe being disposed on a rear surface of a piezoelectric element employed in the ultrasonic probe to attenuate ultrasonic waves radiated from the rear surface of the piezoelectric element, the backing member comprising a mixture of a base member and a filler,
wherein the base member is a polyurethane resin, and
the filler is manganese powder.

2. The backing member of claim 1, wherein a proportion of the manganese powder is in a range of 40 wt % to 90 wt % to obtain an acoustic impedance of 3 to 5 MRayls.

3. An ultrasonic probe comprising a backing member, a piezoelectric element fixed to the backing member, an acoustic matching layer fixed to the piezoelectric element, and an acoustic lens fixed to the acoustic matching layer, wherein the backing member is the backing member according to claim 1.

4. An ultrasonic probe comprising a backing member, a piezoelectric element fixed to the backing member, an acoustic matching layer fixed to the piezoelectric element, and an acoustic lens fixed to the acoustic matching layer, wherein the backing member is the backing member according to claim 2.
